(19) United States
(12) Patent Application Publication
(10) Pub. No.: US 2005/0009046 A1
(43) Pub. Date: Jan. 13, 2005

(54) Identification of haplotype diversity

(76) Inventors: Francisco M. De La Vega, San Mateo, CA (US); Hadar I. Isaac, Los Altos, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/804,586

(22) Filed: Mar. 19, 2004

Related U.S. Application Data
(60) Provisional application No. 60/482,249, filed on Jun. 24, 2003.

(51) Int. Cl. C12Q 1/68; G06F 19/00; G01N 33/48; G01N 33/50

(52) U.S. Cl. 435/6; 702/20

(57) ABSTRACT

A numerical approach used to select a reduced subset of single nucleotide polymorphisms (SNPs) from a larger superset and useful for efficiently identifying haplotype blocks or other genetic loci. In general, the methods may be configured to select for the reduced SNP subset with little or no loss of haplotype diversity information. The methods may also be adapted to operate in a more aggressive mode to further reduce the SNP set while maintaining diversity of haplotype blocks with minimal loss of information. Computation of the reduced SNP subset is generally rapid and the methods perform well even when applied to large data sets spanning significant genomic distances.

### CLAIM OF PRIORITY

This U.S. patent application is a non-provisional application of and claims priority to U.S. Provisional Patent Application No. 60/482,249 entitled “Informative SNP Selection Using Block-Free Analysis and Dynamic Programming” filed Jun. 24, 2003 which is hereby incorporated by reference.

### BACKGROUND

1. Field

The present teachings generally relate to the field of genetic analysis and more particularly to a system and methods for haplotype analysis using a data reduction approach.

2. Description of the Related Art

Single nucleotide polymorphisms (SNPs) are one of the most abundant forms of genetic variation in biological organisms. It has been determined that single nucleotide changes occur with an approximate frequency of one in every 500 base pairs in the human genome. Detailed analysis of SNPs has proved useful in a variety of biological applications including susceptibility mapping of mutations that contribute to complex diseases.

Linkage disequilibrium (LD) arises from groupings of SNPs which are found to be present across relatively large genetic distances and may be correlated to specific populations. Detailed evaluation of LD mappings indicate that reduced sets of contiguous chromosomal segments or haplotype blocks exist wherein the diversity of a selected haplotype is generally restricted to a small subset of possible SNP combinations. Like SNP identification, detailed haplotype analysis can provide useful information in various disease and pharmacogenomic studies.

Frequently, when SNPs are initially selected for haplotyping analysis, relatively little is known about the existence or location of LD blocks, nor about the number and relative frequencies of haplotypes within the blocks. Conventional approaches typically sample large numbers of bases in selected chromosomal regions under study in an attempt to aid in haplotype identification. This “over-sampling” approach is inefficient, time-consuming, and expensive. Furthermore such an approach may be impractical to conduct when examining large sample populations. Consequently, it is desirable to devise a manner in which the quantity of information to be evaluated during haplotype analysis is reduced while at the same time maximizing the amount of useful information that can be obtained from the analysis.

### SUMMARY

In various embodiments the present teachings describe a system and methods for performing SNP analysis and haplotype identification using a data reduction approach in which a reduced subset of SNPs required for capturing haplotype diversity is utilized. In one aspect, application of these methods enable discrimination of common haplotypes present within an SNP block without significant loss of information. Furthermore, using a more aggressive approach, the haplotype block size can be further reduced while still maintaining a relatively high percentage of the original haplotyping information. The disclosed methods are useful in reducing the quantity of information associated with performing detailed haplotyping analysis and desirably improve the efficiency with which subsequent downstream applications may be performed.

In one aspect, the present teachings describe a method for analyzing nucleotide sequence information during haplotyping analysis. This method further comprises the steps of: (a) selecting a data superset comprising single nucleotide polymorphism (SNP) information describing a plurality of SNPs, each SNP associated with a plurality of haplotypes, wherein each haplotype is determined by the sequence of SNPs present in the haplotype; (b) identifying groupings of analogous SNPs from the data superset whose sequences are analogous in two or more haplotypes; (c) selecting at least one representative SNP from each grouping of analogous SNPs to be included in a reduced data subset; and (d) performing a haplotyping analysis using the reduced data subset.

In another aspect, the present teachings describe a method for analyzing nucleotide sequence information comprising the steps of: (a) selecting a data superset comprising single nucleotide polymorphism (SNP) information describing a plurality of SNPs, each SNP associated with a plurality of haplotypes, wherein each haplotype is determined by the sequence of SNPs present in the haplotype; (b) identifying regions of analogous SNP information for each of the plurality of haplotypes; (c) identifying at least one representative SNP from the analogous SNP information for each region; (d) forming a reduced data subset wherein at least a portion of the analogous SNP information is excluded from the reduced data subset while haplotype diversity is preserved by inclusion of the at least one representative SNP in the reduced data subset; and (e) performing the haplotyping analysis using the reduced data subset.

In still another aspect, the present teachings describe a system for analyzing nucleotide sequence information during haplotyping analysis. This system comprises: a data collection component that provides functionality for selecting a data superset comprising single nucleotide polymorphism (SNP) information describing a plurality of SNPs, each SNP associated with a plurality of haplotypes, wherein each haplotype is determined by the sequence of SNPs present in the haplotype; a first data analysis component that provides functionality for identifying a plurality of diversity subsets, each comprising one or more SNPs associated with a selected haplotype, by selecting combinations of SNPs associated with the selected haplotype; a first computational component that provides functionality for calculating an entropy value for each diversity subset and comparing the resulting entropy values to an entropy value determined for the diversity subset containing substantially all associated SNPs; a second data analysis component that provides functionality for identifying an refined diversity subset from the data superset having substantially the greatest entropy value and least number of associated SNPs; and a second computational component that provides functionality for performing the, haplotyping analysis using the refined diversity subset.

In a further aspect, the present teachings describe a method for analyzing nucleotide sequence information during haplotyping analysis comprising the steps of: (a) selecting a data superset comprising single nucleotide polymorphism (SNP) information describing a plurality of SNPs, each SNP associated with a plurality of haplotypes, wherein each haplotype is determined by the sequence of SNPs present in the haplotype; (b) performing a first data reduction on the data superset by identifying redundant SNPs comprising two of more SNPs whose sequences are identical or complimentary for each of the plurality of haplotypes and removing at least a portion of the redundant SNPs from the data superset; (c) performing a second data reduction on the data superset by comparing the SNP information in a pairwise manner to identify analogous SNPs whose sequences are identical in two or more haplotypes and removing at least a portion of the analogous SNPs; and (d) performing a haplotyping analysis using the remaining SNP information in the data superset.

### BRIEF DESCRIPTION OF THE DRAWINGS

FIG. 1 illustrates an exemplary haplotype block diversity distribution.

FIG. 2 illustrates an exemplary haplotype/allele state matrix used to perform SNP data reduction.

FIG. 3 illustrates a method for conducting haplotyping analysis wherein a lossless mode and lossy mode data reduction approach are applied.

FIG. 4A illustrates a method for conducting lossless mode data reduction.

FIG. 4B illustrates an exemplary haplotype/allele state matrix organization of SNP information during the lossless mode data reduction.

FIG. 5A illustrates an exemplary application of lossless mode data reduction in conjunction with a first phase of the haplotype/SNP allele state matrix.

FIG. 5B illustrates an exemplary application of a second phase of the lossless mode data reduction in conjunction with a haplotype/SNP allele state matrix.

FIG. 6A illustrates a method for conducting lossy mode data reduction.

FIG. 6B illustrates an exemplary application of a lossy mode data reduction in conjunction with a haplotype/SNP allele state matrix.

FIG. 6C illustrates the resulting haplotype/SNP allele state matrix following application of the lossless approach.

FIG. 6D illustrates a table of SNP subsets following application of the lossy mode data reduction.

FIG. 7 illustrates another data reduction method for use in haplotyping analysis in which locally and globally optimal solutions are considered.

FIG. 8 illustrates the performance of the lossless and lossy data reduction approaches as applied to an actual sample data set.

FIG. 9 illustrates an exemplary state matrix and associated data used in determining a globally optimal solution for the reduced SNP subset.

FIG. 10 illustrates a distribution of SNP subsets for an exemplary data set used in evaluating the data reduction methods.

### DETAILED DESCRIPTION OF CERTAIN EMBODIMENTS

The present teachings describe an analysis approach in which a subset of single nucleotide polymorphisms (SNPs) are selected from a larger principle SNP set (superset), representing haplotype block information or other genetic loci, to provide substantially similar information regarding sequence diversity while at the same time reducing the total quantity of information to be processed. In various embodiments, identification of the SNP subset desirably reduces the computational demands associated with evaluating large quantities of haplotyping information by eliminating redundant or non-informative SNP information thereby decreasing the analysis complexity and improving efficiency.

In one aspect, the disclosed methods may be adapted to a computerized analysis platform or software application wherein the analysis is performed in a substantially automated manner. As will be described in greater detail hereinbelow, automated data analysis may be performed using a multi-step approach whereby the principal SNP set is arranged so as to provide rapid elimination of a first category of SNPs efficiently reducing the overall quantity of data to be evaluated in determining the final SNP subset. This data reduction approach desirably improves computational efficiency by reducing the number of SNPs which will be subsequently evaluated by more stringent and computationally demanding criteria.

SNP Correlation and Haplotyping

The significance of haplotype identification stems from the widespread presence of SNPs throughout the human genome. SNPs generally display a bi-allelic property wherein only two different alleles are typically encountered for a selected genomic position. A so-called major allele is generally present in the majority of chromosomes in a population, and its alternative variant, a minor allele, is generally present with a lesser frequency of occurrence. While most SNPs are neutral and do not affect phenotype, they can be used as surrogate markers for positional cloning of genetic loci, because of the allelic association, known as linkage disequilibrium (LD), that can be shared by groups of adjacent SNPs. In one aspect, LD forms the basis for haplotype block identification wherein a plurality of SNPs are grouped together as occurring with a greater frequency than would be expected through chance alone.

As an example of a simplified linkage disequilibrium calculation, a major allele or SNP “A” and a minor allele or SNP “a” may be designated for a selected position in a genome (locus). An individual may have either the major or minor allele with the frequency of occurrence of the major allele “A” in the population identified as f_{A} and the frequency of occurrence of the minor allele being fₐ=1−f_{A}. Likewise for a second SNP, a major and minor allele may be identified as “B” and “b” respectively with corresponding occurrence frequencies of f_{B} and f_{b}=1−f_{B}.

Assuming random combination without correlation is responsible for the presence of each allelic variant in a selected population it can be established that the probability of occurrence of both traits within a selected organism can be reflected as one of the frequency products: f_{AB}=f_{A}f_{B}; f_{Ab}=f_{A}f_{b}; f_{aB}=fₐf_{B}; f_{ab}=fₐf_{b} However, as previously noted, many SNPs occur with at least some degree of correlation (disequilibrium state), consequently, the probability of occurrence of both traits can be reflected as one of the frequency products: f_{AB}=f_{A}f_{B}+D; f_{Ab}=f_{A}f_{b}−D; f_{aB}=fₐf_{B}+D; f_{ab}=fₐf_{b}−D where D is the measure of disequilibrium. Finally, from the above information it can be determined that D=f_{AB}−f_{A}f_{B}.

LD is eroded by gene conversion and recombination and the amount of LD may depend on the relative age of the mutations and on the demographic history of the population. The extent of LD across a genomic region also generally dictates the density of SNP markers necessary to ensure association between a marker and a causative allele sought.

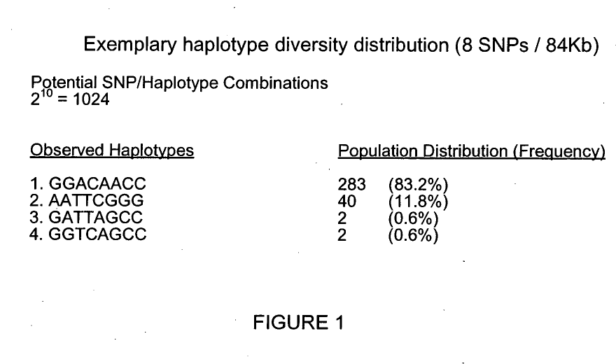

LD distances may be relatively short, extending only a few kilobases (Kb) or less or may be much larger ranging from 5 Kb to 60 Kb or more. In many instances, LD patterns across a genomic region may appear as a series of one or more LD “blocks” which show little evidence of recombination and suggest that a reduced set of contiguous chromosomal segments, or haplotypes, exist in specific populations. For example, as shown in FIG. 1 for an exemplary haplotype block spanning 84 Kbs for which there are 10 associated SNPs, 2¹⁰ or 10²⁴ theoretically possible SNP combinations/haplotypes exist. Despite this large number of candidate haplotypes as is generally observed only a small number of actual haplotypes comprise the vast percentage of the haplotype diversity in a selected population. For example, as shown in FIG. 1, only four observed haplotypes may be present to any significant extent in the population. The frequency with which a selected haplotype occurs may also vary greatly for each observed haplotype with a singular prominent haplotype within the population and others being represented to a lesser degree. Thus, haplotype 1 shown in the exemplary distribution may have a substantially greater frequency of occurrence (83.2%) as compared to haplotypes 2-4 (11.8%−0.6%). It will be appreciated that the aforementioned haplotype characteristics and distributions are meant as examples only and as such other haplotype combinations and distributions may be observed with substantially different values.

LD block patterns typically change depending on the population sampled and because of historical differences; for example, certain populations may show longer LD blocks and less evidence of recombination events than other populations. Generally, the haplotype diversity in a selected population is substantially constant in a particular region irrespective of the number of SNPs sampled; therefore typing an arbitrarily large number of SNPs as is conventionally done within a LD block may be unnecessary and contribute to analytical inefficiencies including increased analysis time and cost. In various embodiments, the present teachings provide an improved manner of SNP and haplotype analysis by selecting a reduced or minimal subset of SNPs within selected LD blocks, or any other discrete genetic locus. This subset of SNPs provides comparable information as the larger SNP set (superblock) from which it originated and enables discrimination of common haplotypes present in a block without appreciable loss of diversity information.

When SNPs are initially selected for typing, generally not much is known about the existence or location of LD blocks, nor about the number and relative frequencies of haplotypes within the blocks. Conventional methods typically address this issue by “over-sampling” the chromosomal region, (e.g. selecting a large number of SNPs to densely cover the region under study). The degree of over-sampling in conventional methods is oftentimes cost-limited when detecting the genotype for each SNP, Consequently, it is desirable to reduce or minimize the number of SNPs used in a particular study or analysis.

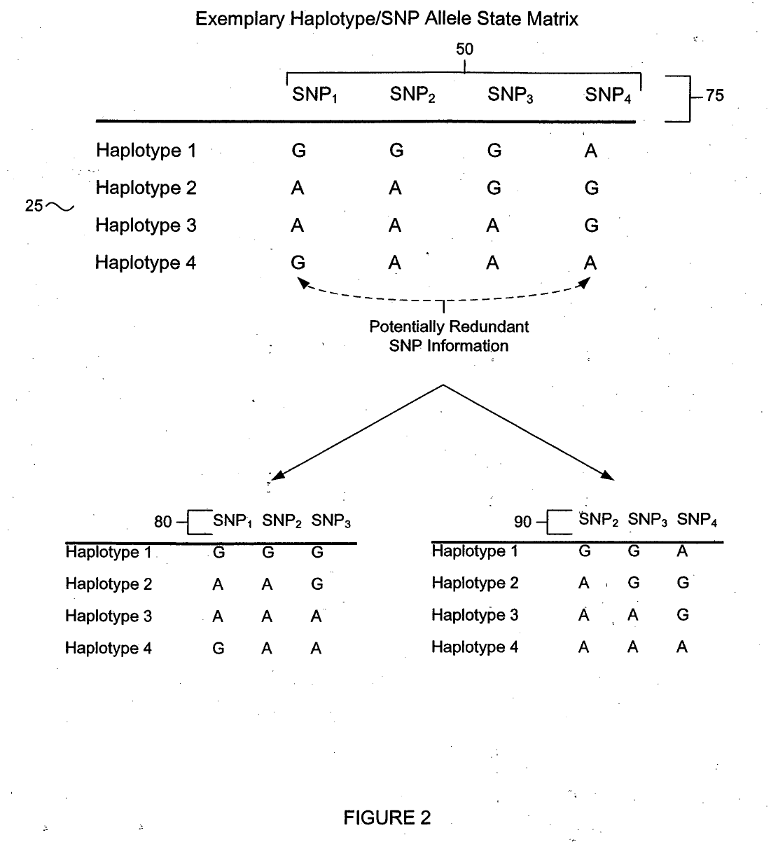

FIG. 2 illustrates the concept of SNP set minimization for a group of four exemplary haplotypes 25. As shown in this figure, each haplotype comprises four discrete SNPs 50 forming a SNP superset 75 that may be used in discriminating between each haplotype on the basis of composition, the sequence of which may vary from one haplotype to another. In one aspect, the exemplary haplotypes 25 represent a common haplotype grouping with their composition and frequency of occurrence representative of the majority or totality of a selected sample population.

As will be described in greater detail hereinbelow, the composition of SNP₁ and SNP₄ can be shown to provide redundant information which does not necessarily contribute substantially to discriminating between the haplotypes present in the grouping. Thus, elimination of SNP₁ or SNP₄ from the SNP superset 75 may yield two SNP subsets 80, 90 wherein the information provided by each SNP subset 80, 90 provides substantially the same haplotype resolving capability as the SNP superset 75 from which they originated. Furthermore, the size of the SNP subsets 80, 90 are smaller than the superset 75 and therefore facilitate more rapid analysis during haplotype identification and analysis. Data reduction in this manner is desirable especially in designing software approaches to data analysis and results in reducing the complexity and time required for analysis.

In various embodiments, the present teachings, desirably identify and exclude potentially redundant SNP information thereby providing an effective means to perform SNP set size reduction. The SNP set size reduction approach applies a novel method of SNP subset identification that implements a multi-step approach to quickly remove “easily” identified redundant SNPs and subsequently applying more rigorous means to further reduce the SNP subset size. This manner of SNP subset reduction desirably improves computational performance by reducing the number of SNPs which are evaluated by the more computationally demanding reduction methods.

As will be described in greater detail hereinbelow, when selecting a population sample large enough to allow for accurate inference of the haplotype distributions, the method can reduce the set of SNPs required for adequate coverage with substantially no loss of haplotype discrimination. Furthermore, using a more aggressive SNP selection approach the method can be used to further eliminate additional SNPs while minimizing loss of haplotype information.

Approach to SNP Set Minimization

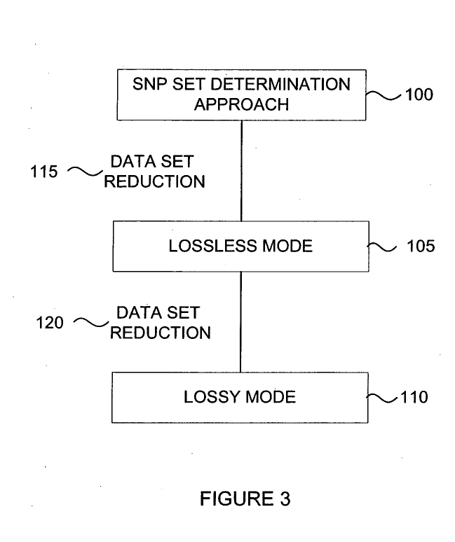

As previously described, the present teachings provide means for data set reduction or minimization during haplotyping analysis. FIG. 3 illustrates a block diagram depicting two principal modes of operation of a SNP set determination method 100 used to general data sets moderated by various degrees of stringency. The method 100 may be generally described as capable of operating in a lossless mode 105 or a lossy mode 110. When operating in lossless mode 105 the method 100 performs SNP data reduction with substantially no loss of haplotyping information while operating with a relatively high degree of operational efficiency. In lossy mode 110 the method 100 performs SNP data reduction in a more rigorous or aggressive manner to further reduce the quantity of SNPs in the data set to be used in haplotyping analysis. As shown in FIG. 3, the lossless mode 105 of SNP data reduction may be used to generate a first SNP data set reduction 115 that serves as input for the lossy mode 110 which subsequently generates a second more stringent SNP data set reduction 120. As will be described in greater detail hereinbelow, the sequential manner of data set reduction from lossless mode 105 to lossy mode 110 desirably provides a means to efficiently reduce the size of the data set using the computationally rapid lossless mode 105 prior to performing the more computationally demanding lossy mode 110 reduction. While the exemplified sequential nature of operation of these two approaches generally produces high quality reduced SNP data subsets, it will be appreciated that these methods may be operated independently of one another to achieve different degrees of SNP subset stringency. For example, if it is desirable to maintain substantially all of the haplotyping diversity for a selected sample set, the lossless mode 105 may be used alone without further processing in the lossy mode 110. Alternatively, if a higher stringency SNP data set is desirable, then the lossy mode 110 may be used either in conjunction with or without operation of the lossless mode 105. The lossy mode reduction may also be configured to generate data sets based on the amount of haplotyping diversity loss which can be tolerated. For example, the lossy approach can be configured such that substantially no diversity is lost when generating the reduced data set or a threshold can be identified as being the amount of haplotyping diversity which should desirably be retained following data reduction. Additional details for selecting the threshold value of desired diversity retention will be described in greater detail hereinbelow.

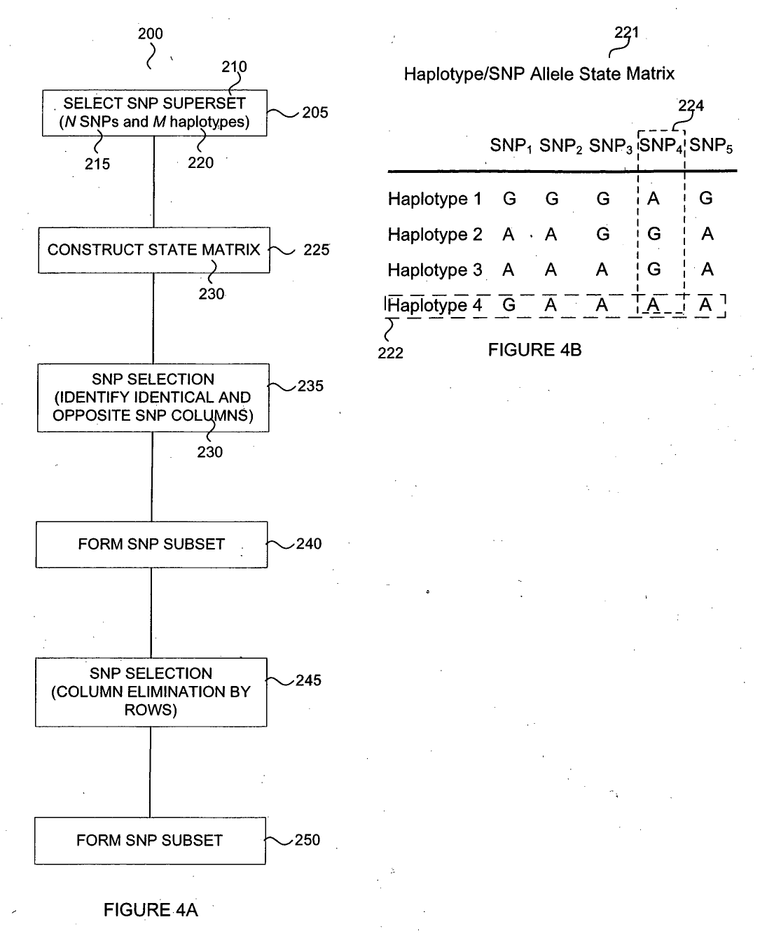

FIG. 4A details the operations performed in one lossless mode approach 200. The method 200 comprises two operational phases each designed to remove a portion of applicable information relating to the haplotype/allele information to be evaluated. In various embodiments, and for the purposes of illustration, the haplotype/allele information may be arranged in a state matrix configuration 221 (shown in FIG. 4B) wherein a plurality of associated haplotypes form rows 222 of the matrix 221 and constituent SNPs form columns 224 of the matrix 221.

The method 200 commences in state 205 wherein a SNP superset 210 comprising a plurality of “N” SNPs 215 associated with a plurality of “M” haplotypes 220 is selected. The SNP information used as input for the method 200 may originate from many sources including but not limited to: experimental sequence information, reference sequence information, and SNP database information. The organization and format of the SNP data need not conform to a particular standard and may be arranged as is convenient for the investigator to identify one or more haplotype blocks which will undergo evaluation.

In state 225, the haplotype/SNP allele state matrix 230 is defined using the SNP superset 210. It will be appreciated that the state matrix representation of data is but one of many possible means by which the haplotyping data may be arranged. While the principals of operation of the method 200 are directed towards a data configuration which adopts the matrix arrangement it will be appreciated that other data configurations and arrangements can be used which will also produce suitable results in data set reduction. As such, these alternative forms configuring or arranging the data are considered but other embodiments of the present teachings.

In state 235 the first phase of the lossless method 200 commences with the identification of columns 224 that comprise SNP information identical to or opposite of another column. For example, as shown in FIG. 4B the column associated with SNP₁ possesses an SNP composition that is opposite that of the column associated with SNP₄. Likewise, the column associated with SNP₂ possesses an SNP composition that is identical to that of the column associated with SNP₅.

In one aspect, a column that is identical (or complimentary) to another column represents a SNP whose behavior is substantially identical to another SNP for each sample evaluated. Redundant SNP information such as this does not generally provide any more useful information beyond the first SNP identified as having a particular haplotype block sequence behavior. As such, only a single SNP need be retained in the SNP subset generated in state 240 which may be used to represent the characteristics of the group of SNPs which behave substantially identically to one another. In a similar manner, a SNP column that exists as an opposite of another column may be identified as a SNP whose behavior is predictable from the behavior of another SNP by inversion of its sequence. Consequently, one or more SNPs having opposite haplotype block sequence behavior as compared to another SNP do not generally provide new information and may also be excluded when forming the SNP subset in state 240.

The SNP subset formed in state 240 therefore represents the collection of those SNPs selected from the initial SNP superset 210 having discrete haplotype sequence behavior which is neither identical to, nor opposite of, other SNPs in the SNP subset. Applying this to approach to the N columns of the exemplary matrix therefore reduces the matrix to N′ substantially unique columns where N′<N.

In state 245, a second data reduction approach is performed wherein each SNP column is evaluated against the haplotype rows. Any SNP column whose removal from the SNP subset does not reduce the number of unique rows may be excluded to from a refined SNP subset. In one aspect, each row is representative of the allelic states of the SNPs for a specific haplotype. Removing a “useful” SNP (one which uniquely identifies a particular haplotype) may affect the ability to detect at least one haplotype in the sample population. In such a case, two of more haplotypes would be associated with the same allelic states using the remaining SNPs of the subset, thereby reducing the number of unique rows. Therefore, if the exclusion of a column does not reduce the number of unique rows, the associated SNP information can be withheld from the refined SNP subset without loss of haplotyping information.

In one aspect, SNP subset selection according to the data reduction approach of state 245 may be implemented independently of the SNP subset selection according to the data reduction approach of state 235. One rational for this is that performing data reduction by columnar elimination as described for state 245 readily eliminates data that would have been otherwise eliminated in state 235.

In various embodiments, performing a multi-tiered data reduction as described conveys certain performance benefits wherein the initial data reduction approach associated with state 235 generally operates in a rapid and computationally efficient mode such that the haplotype/SNP data set can be reduced prior to application of data reduction approaches that are more computationally demanding and time-consuming. Thus, the multi-tiered data reduction approach may improve the speed with which the overall analysis can be performed over using a singular data reduction method alone.

Lossless Mode Data Minimization

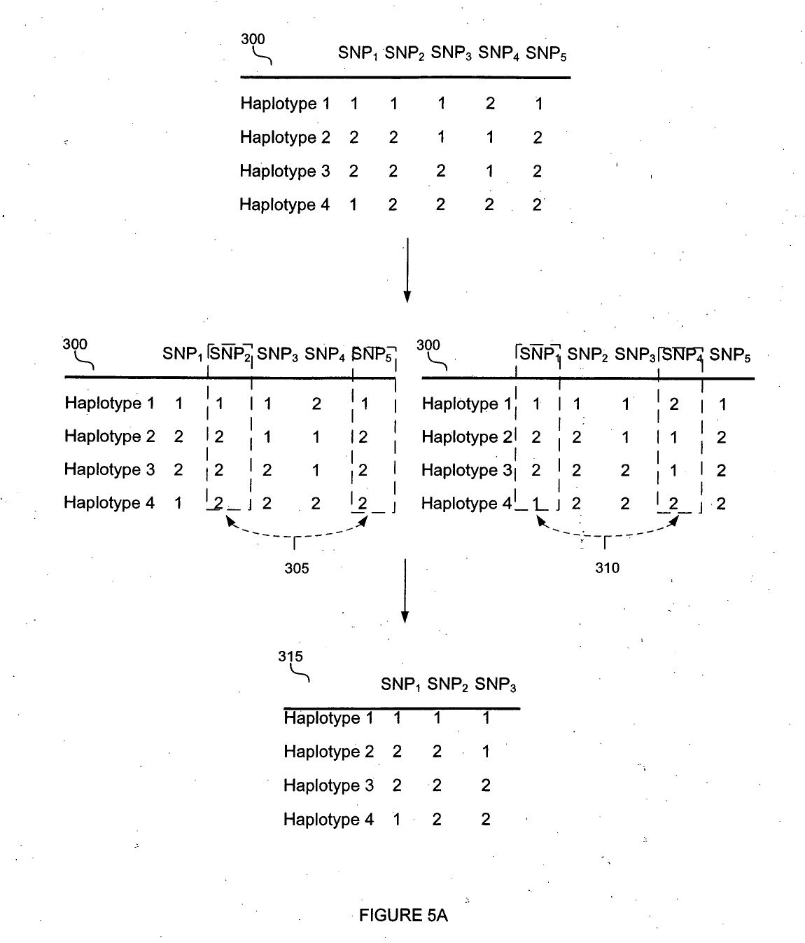

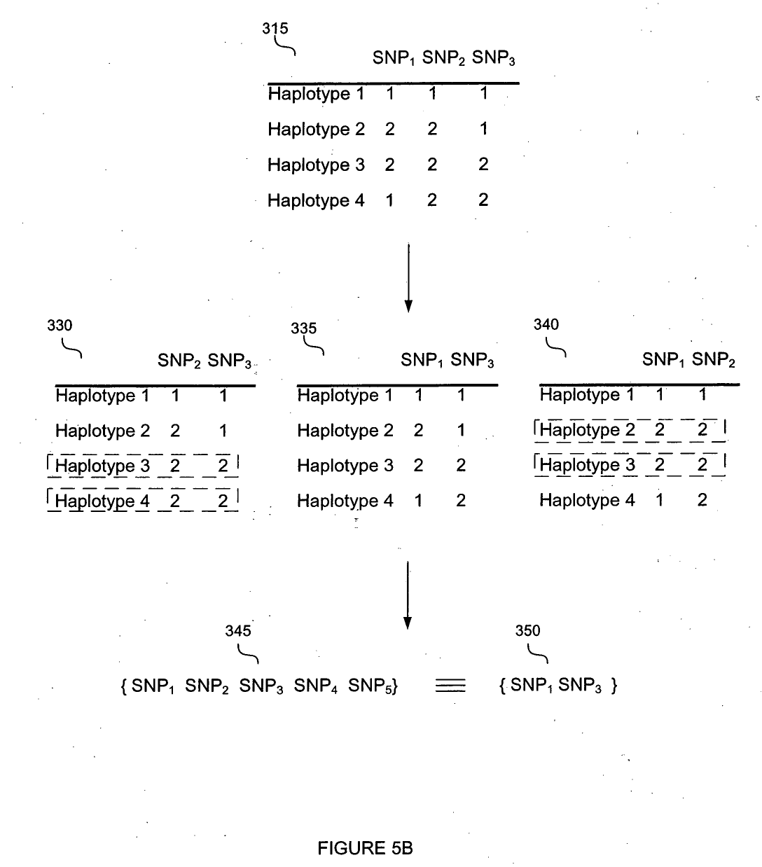

FIG. 5A illustrates application of the aforementioned data reduction methods as applied to an exemplary haplotype/SNP allele state matrix 300. Initially, the exemplary state matrix comprises five SNPs and four haplotypes that yield a plurality of allelic responses. In this illustration the allelic responses are indicated as either “1” or “2” and may be readily represented by other identifiers such as base sequences “G”, “A”, “T”, “C” or the like. Applying the lossless method 200, SNP selection according to the first data reduction approach may be used to identify two pairs of SNPs columns having similar allelic responses. A similar allelic response pattern 305 is shown for SNP₂ and SNP₅ wherein either SNP₂ or SNP₅ may be excluded from further analysis are representing redundant information. Likewise, an opposing response pattern 310 is shown for SNP₁ and SNP₄ wherein either SNP₁ or SNP₄ may be excluded from further analysis. Performing the above-indicated operations may yield reduced data set comprising a haplotype/SNP allele state matrix 315 having only 3 SNP columns as compared to the original 5 SNP of the state matrix 300 from which it was derived.

The preceding description represents one possible series of operations associated with the first data reduction approach 235. Using the resultant haplotype/SNP allele state matrix 315, the method 200 may then proceed to the second data reduction approach 245. As shown in FIG. 5B, the haplotype/SNP allele state matrix 315 may be arranged as a series of sub-matrices 330, 335, 340 representative of pairwise comparisons between selected SNPs. When evaluating these matrices 330, 335, 340 according to the second data reduction approach 245 it can be determined that the first and the third matrices 330, 340 each contain a row which duplicates another within the selected matrix. Conversely, the second matrix does not contain any row duplication and consequently this SNP set may be considered minimized with respect to potential data reduction.

Using the aforementioned sub-matrix information, it can be determined that SNP₂ can be eliminated with no loss of haplotype detection. The resulting data subset 350 therefore comprises SNP₁ and SNP₃ which provides substantially the same haplotype detection ability as the full set 345 (SNP₁, SNP₂, SNP₃, SNP₄, and SNP₅).

The aforementioned example illustrates that the first phase reduction may result in 2 less SNPs needed for full diversity capture and the second phase reduction may result in a still further subset reduction of 1 SNP. In various embodiments, this final SNP subset 350 may be considered a minimized SNP subset according to the lossless approach 200 wherein substantially no haplotype diversity information is lost while at the same time reducing the amount of information contained in the SNP subset by a significant amount. It will be appreciated that the aforementioned methods may be applied to other haplotype/SNP information to yield similar minimized data sets. Furthermore, each phase of data reduction may result in the elimination or exclusion of one or more SNPs from the initial data set or possibly no SNPs in the case where the input haplotype/SNP information is already minimized with respect to the particular reduction phase being applied.

In various embodiments, the aforementioned lossless mode data minimization approach is generally directed towards producing a SNP data set that contains the least amount of information necessary to provide for complete haplotyping analysis with little or no loss of haplotyping diversity. Generally, this method operates under the criteria that the haplotype list be exhaustive and that the SNP population be large enough to allow for accurate inference of the haplotype distributions. When these criteria are met the data reduction approach is expected to perform well.

Lossy Mode Data Minimization

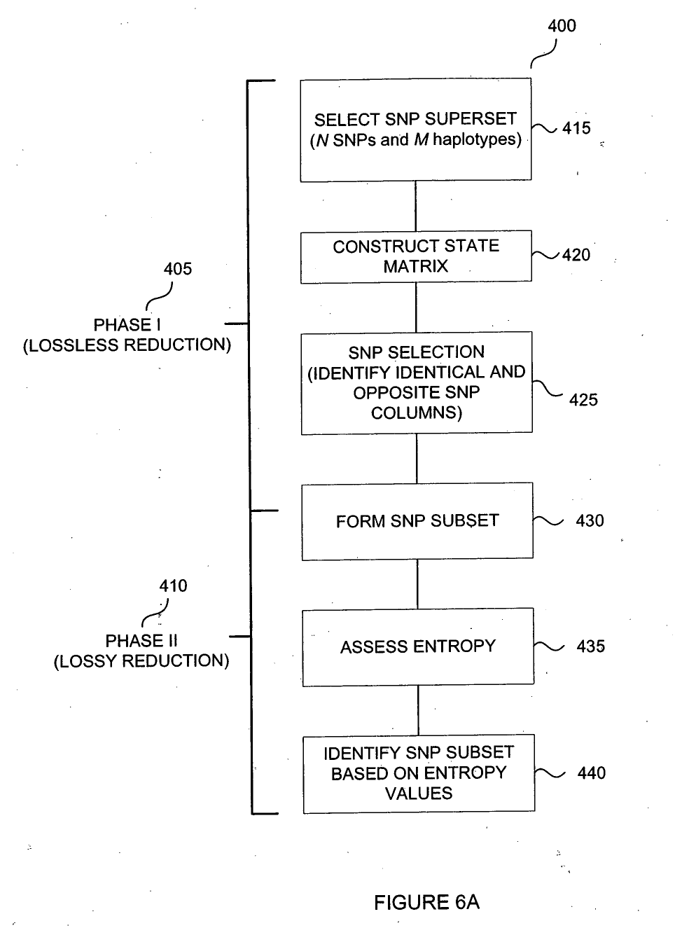

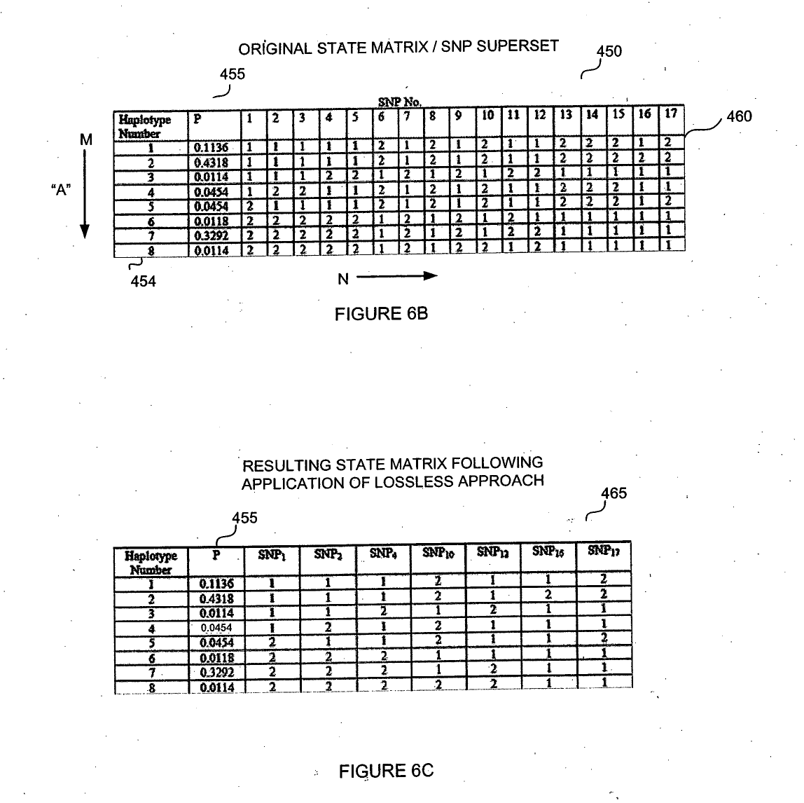

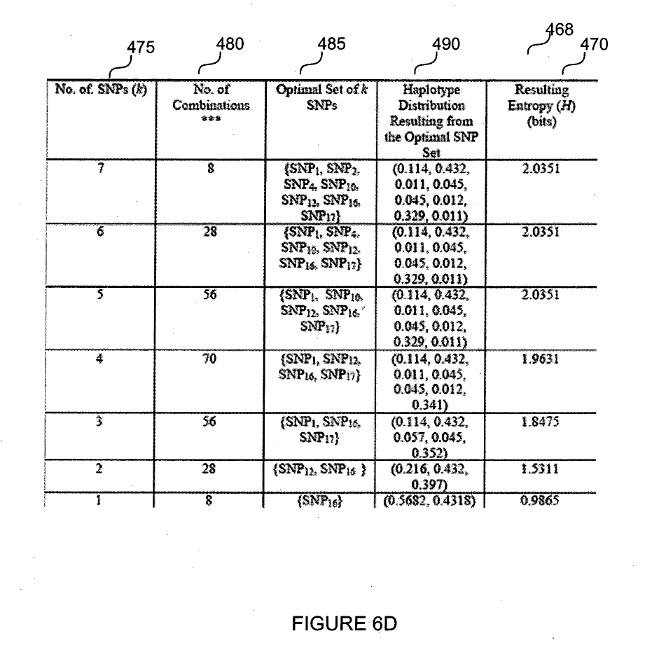

In various embodiments, it may be desirable to attempt to reduce the SNP data set beyond that which may be possible using only the lossless approach. For example, if budget constraints are tight or cost minimization is a factor, it is still generally desirable to retain a high degree of haplotype diversity information although some degree of loss may be tolerable. In such instances, the lossy mode data minimization approach may yield improved results over that of the lossless mode approach. One possible means for performing a lossy mode reduction is detailed below using a similar state matrix as previously introduced in conjunction with the lossless mode reduction. FIG. 6A illustrates a method 400 for performing a lossy mode data reduction used in conjunction with an exemplary haplotype/SNP allele state matrix 450 shown in FIG. 6B, the state matrix 410 may again be defined by a plurality of haplotype blocks comprising “N” SNPs and “M” haplotypes for which a probability vector “P” 455 having a length or value M is assigned. According to this data arrangement, Pᵢ may further be defined as the relative associated frequency of occurrence of the i^{th} haplotype. The state matrix “A” 450 therefore comprises a matrix of N columns and M rows, where Aᵢⱼ, (the i^{th} row of the j^{th} column of the matrix A) indicates an allele state (exemplified as “1” or “2” in the illustration) of the j^{th} SNP for the i^{th} haplotype.

Referring again to FIG. 6A, in various embodiments, the method 400 may be subdivided into two principal phases 405, 410. The first phase 405 comprises a lossless mode data reduction similar to that described previously wherein a SNP superset is selected in state 415, a state matrix is constructed using the identified haplotype/SNP allele information in state 420 and an SNP subset is generated by removal of redundant SNP information in state 425. In one aspect, removal of redundant SNP information proceeds in a manner similar to the lossless approach wherein columns in the state matrix are compared and SNP information which is identical to or opposite of other SNP information is excluded from subsequent analysis. This manner of data reduction, prior to the lossy approach described below, desirably serves to reduce the haplotype/SNP data set prior to application of more rigorous (e.g. computationally demanding/time consuming) methods. It will be appreciated that the resultant reduced data subset formed in state 430 may optionally comprise the original haplotype/SNP allele information or a portion thereof which has been processed by other means prior to application of the lossy approach.

In various embodiments, application of the initial data reduction is desirable to remove redundant SNP information while preserving a substantial amount of the information contained in the probability vector P. For the purpose of quantifying the information in the probability vector P, a Shannon Entropy determination approach may be used as defined by the equation:

$$Equation 1 : \\ H = - {\sum }_{i = 1}^{M} {P}_{i} ln ( {P}_{i} )$$

As will be described in greater detail hereinbelow, entropy measurement according to this model may be used to evaluate the SNP information to determine how many bits of information are present on average for a selected data set. Based in part on this manner of interpreting entropy, the method 400 proceeds to the second phase 410 of the analysis wherein the entropy (H) is computed in state 435 for the probability vector P arising from

$$( \frac{{N}^{′}}{k} )$$

possible selections of k SNPs. In state 440, the selection having substantially the greatest entropy value is chosen as the desired selection which generally possesses a reduced quantity of SNP data while still preserving a significant portion of the haplotyping diversity, discrimination and identification information. Additional details of the operation of the lossy mode data reduction 410 are described below in connection with the exemplary matrix 450 illustrated in FIG. 6B.

According to the lossy mode data reduction 410, by selecting k out of N′ SNPs, N′−k columns may be eliminated. The resulting matrix, having k columns, may have fewer unique rows than the full matrix, having N′ columns. In the instance where a row is repeated more than once, it may be determined that there are several “minor” haplotypes that have been measured as a single “major” haplotype. Such an occurrence may arise as a result of having fewer SNPs present in the data set resulting in some degree of loss of haplotype diversity. The relative frequency (e.g. probability) of the “major” haplotype can be determined to be the sum of the frequencies of the “minor” haplotypes. Thus, when a data reduction of SNP columns results in repeating haplotype rows, the repeating rows can be combined into a single row, and their respective probabilities summed to form a new probability. Consequently, the vector P will be shorter, have a larger associated value, and reduce the calculated value of the entropy, H.

By applying the aforementioned approach, the combination having substantially the smallest reduction of entropy may be deemed to be the optimal selection. It will be appreciated that if all the rows are unique after elimination of N′−k columns, the entropy will not be reduced and k SNPs may be used with no loss of information, as in the lossless approach.

The exemplary data shown in FIG. 6B used to illustrate the operation of the lossy approach 410 represents a LD block defined by the haplotypes/SNP allele state matrix 450 obtained from Chromosome 6, overlapping the Human gene TTK (RefSeq ID NM_{—}003318, Celera ID hCD401205). This LD block 450 comprises 17 SNPs with 8 inferred haplotypes as determined by conventional methods. The distribution of haplotypes 454 within the LD block 450 further comprises 2 major haplotypes “2” and “7” with approximate frequencies of occurrence of 43% and 33% respectively. The remaining diversity is distributed among the other 6 haplotypes. The allelic states 460 of the 17 SNPs and their respective probabilities for each of the 8 haplotypes therefore serve as the basis for creating the SNP superset and associated information which is used by the method 400.

Following application of the lossless approach 405, a reduced state matrix 465 may be identified as shown in FIG. 6C. Here the number of SNPs in the reduced state matrix 465 is diminished significantly from 17 candidate SNPs down to 7 SNPS. The SNP subset which forms the resulting state matrix 465 comprises SNP₁, SNP₂, SNP₄, SNP₁₀, SNP₁₂, SNP₁₆, and SNP₁₇ as shown. At this point, all of the haplotype diversity information is preserved, including their distribution, with the entropy of the original distribution of haplotypes being approximately H(P)=2.0351 bits. From here, the lossy approach 410 may be utilized to reduce the exemplary state matrix 465 further.

Various subsets of SNPs which result from application of the lossy approach 410 are illustrated by the SNP combination chart 468 shown in FIG. 6D. The subsets are arranged according to the number of SNPs 475 contained in the subset and identify the optimum SNP subset for k SNPs out of the 8 SNPs that passed the lossless data reduction 405. The results indicate that haplotype information is fully preserved for the subsets of 7, 6, and 5 SNPs (determined by comparing the resulting entropy value 470 for the SNP subset to the entropy value calculated for the original distribution of haplotypes. Thus, in various embodiments, the lossy approach 410 can be used with a selected degree of stringency that does not impact the overall haplotype diversity.

If a higher degree of stringency is desired, other SNP subsets may be selected whose resulting entropy value 470 is less than that of the superset from which it was derived. As shown in the illustration, for a SNP subset size of “4” the resulting entropy value 470 is lower than that of the original SNP superset and some diversity information may be removed to achieve a more manageable (e.g. smaller) SNP subset size. In comparing the entropy values for this SNP subset it can be determined that only a 3.5% loss of entropy is observed as compared to the original haplotype distribution. Likewise, for a SNP subset of size “3” a loss of 9.2% of the original entropy is observed. Using this information, an investigator may select the SNP subset which provides a suitable balance between haplotyping diversity and subset size. Furthermore, analyzing the smallest SNP subsets can be useful in determining which SNPs are most prevalent in a selected population. For example, if the lossy approach 410 is used to completely decompose a data set to a single SNP, this SNP can be inferred to be the most frequent or common SNP in the data set. Such information may also be of value when determining the order and quantity of SNPs to analyze in subsequent investigations.

It will be appreciated that the aforementioned entropy determination approach to data set reduction is but one of numerous possible manners in which haplotype diversity may be determined, it is conceived that the methods described herein need not be limited solely to this diversity metric and that other metrics may also be adapted for used with the data reduction methods of the present teachings. As such, use of diversity metrics other than entropy are considered to be but other embodiments of the present teachings.

Modified Lossy Approach

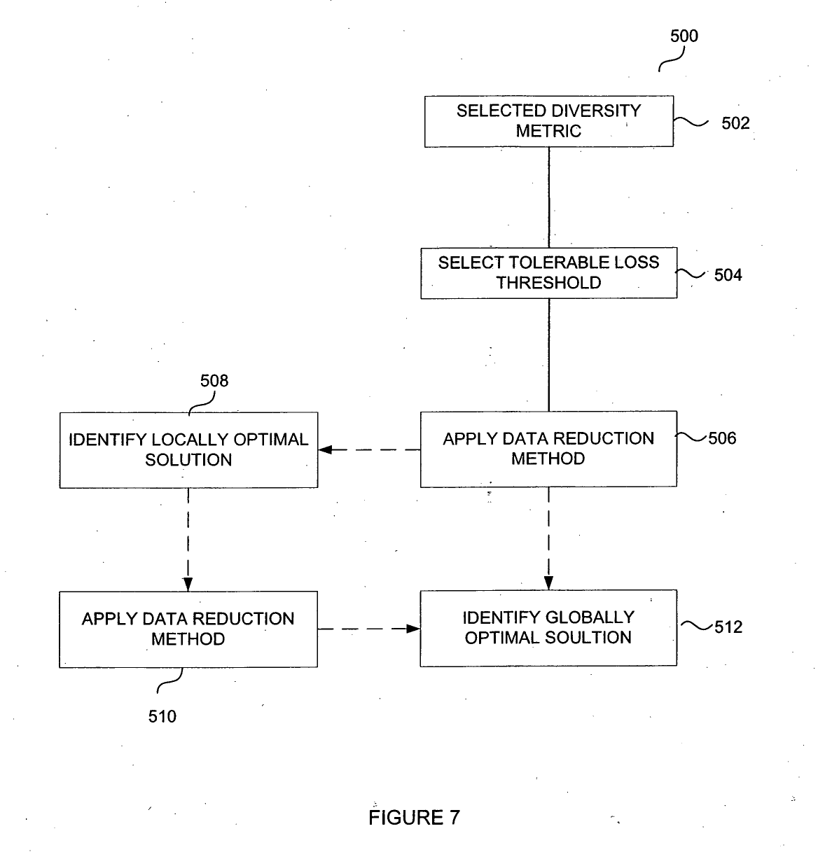

In one aspect, a modified approach to lossy analysis can be conducted as show in FIG. 7. In this method 500, data reduction initially proceeds by selecting a desired haplotype diversity metric 502. As previously described the metric used to evaluate the data can be selected from a number of different approaches including for example entropy determination. In state 504 a determination is made as to how much haplotype or diversity information loss is tolerable (e.g. haplotypes loss threshold). This determination is used to set a threshold for subsequent data reductions in which loss of diversity may result and may further be used to identify which SNP subsets should be selected. Using the identified threshold as a guide, the data reduction methods are applied and the minimum SNP subset is identified in state 506 which achieves the desired loss criteria.

In one aspect, the complexity of data reduction can be evaluated from a combinatorial standpoint. For example, given N SNPs, each SNP can either be included in the reduced data set or excluded from the data set. This gives rise to 2^{N} possibilities which becomes 2^{N}−1 possibilities if the case where all SNPs that are not included are excluded.

The aforementioned analysis of data reduction can be viewed in another way where an optimal solution may be determined to have exactly K SNPs. Thus for each K there are

$$( \frac{N}{k} )$$

potential solutions and thus

$${\sum }_{K = 1}^{N} ( \frac{N}{k} ) = {2}^{N} - 1$$

possibilities can be deduced.

The aforementioned SNP elimination rules corresponding to the lossless mode and lossy mode data reductions can further be applied to a selected data set. In one aspect, the lossless approach may be defined for an allele state matrix as elimination by columns wherein any column that is identical to another column, or is the exact opposite of another column, can be eliminated with no loss of haplotyping diversity. Furthermore, the lossy approach may be defined for an allele state matrix as elimination by rows wherein any column whose elimination does not reduce the number of unique rows can be eliminated. Using the aforementioned rules, a globally optimal solution is selected wherein the lossless approach reduces the data set N to the smaller data set N′ and from the

$$( \frac{{N}^{′}}{k} )$$

possible selections of K SNPs the lossy method determines the selection with the highest haplotype diversity.

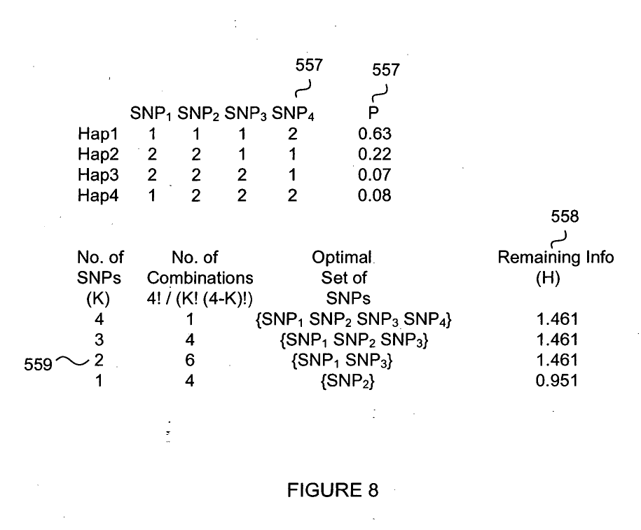

FIG. 8 illustrates an exemplary state matrix 557 and associated probability values 558 for determining an appropriate SNP subset. When applying the aforementioned methods for determination of a globally optimal solution, it can be seen that the SNP subset can be reduced to a size of “2” with no loss of entropy 558 as compared to SNP other larger subsets. Furthermore, as shown, the globally optimal solution can be determined according to the equation:

$$Equation 2 : \\ ( \frac{N !}{( K ! ( N - K ) ! )} )$$

Here N represents the total number of SNPs available that are used to form the subsets and K represents the number of SNPs within a selected subset. From this equation, the number of combinations of SNPs within each selected subset can be determined wherein the subset with the greatest number of combinations may be used to determine the optimal SNP subset. Thus for the exemplary state matrix 557, the SNP subset 559 having “2” SNPs and a high entropy value substantially the same as that of the other larger SNP subsets may be selected as the reduced SNP subset forming the globally optimal solution.

Another approach to the analysis may comprise developing a locally optimal solution wherein the lossless approach is used to reduce the data set N to the smaller data set N′ and the lossy approach is used to reduce N′ to K_{LocalOptimum} wherein K_{LocalOptimum} reflects the locally optimal solution for a selected data set. Using this approach, the performance of determining the globally optimal solution may be improved by “prescreening” candidate SNP sets to determine approximately where the globally optimal solution will reside. In one aspect, the locally optimal solution is first determined according to the aforementioned lossless and lossy approaches. Subsequently, a globally optimal solution is determined using as a K_{LocalOptimum} solution as a starting point for the lossy analysis in state 510. The rationale for such an approach is that the globally optimal solution will not be expected to have more SNPs than a locally optimal solution and therefore restricting the analysis to those provided by the K_{LocalOptimum} solution will generally result in arriving at the globally optimal solution while requiring less possible SNP combinations to be analyzed.

Exemplary Implementation of Methods

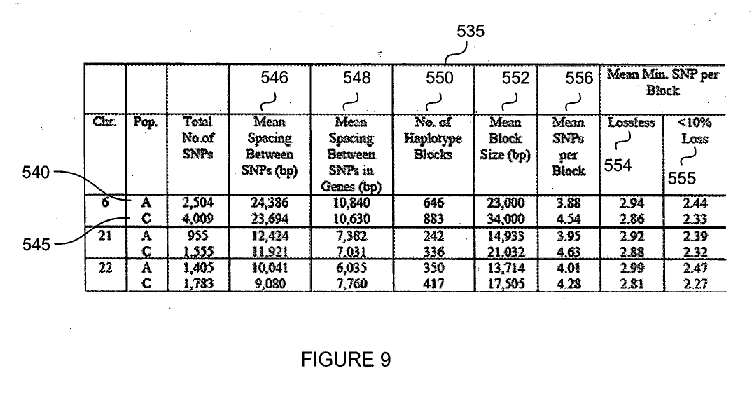

As example of how the aforementioned methods perform in “real-world” contexts, and to assess the utility of each approach, genotyping data from 11,160 SNPs distributed in a gene-centric fashion was evaluated as shown in FIG. 9. For this data, an intragenic spacing averaging of 12 Kb, 8 Kb, and 9 Kb, for chromosomes 6, 21, and 22 respectively was used with LD blocks and haplotypes computed independently for a plurality of populations including 45 African-American and 45 Caucasian DNAs. When considering blocks sizes of 3 or more, 4,864 SNPs formed the African-American population and 7,347 SNPs formed the Caucasian population (generally known to have more and longer LD blocks).

The methods may be implemented in a number of ways and in this example MATLAB® Version 6.1 (The MathWorks Incorporated., Natick, Mass., USA) was used to perform the computations developed by following the aforementioned approaches. The summary of results 535 indicated in FIG. 9 compare an African-American population panel 540 to an Caucasian population panel 545 for the haplotyped blocks detected in the data for chromosomes 6, 21, and 22 described above. Calculations for mean spacing between SNPs 546, mean spacing between SNPS in genes 548, total number of haplotype blocks 550, and mean haplotype block size 552 can be readily determined using any of the aforementioned approaches to data reduction.

As shown by comparing the columns for Mean minimum SNP per block for the lossless approach 554 and the lossy approach 555 to the mean SNPs per block 556 it can be shown that both data reduction methods are able to efficiently reduce the overall data complexity. For example, for Chromosome 6 the mean SNPs per block 556 for African-Americans and Caucasians is 3.88 and 4.54 respectively. These values are reduced significantly to 2.94 and 2.86 when using the lossless approach and still further when applying the lossy methods resulting in values of 2.44 and 2.33 respectively. Thus, the overall data complexity can be reduced by a significant amount using the lossless approach with no expected loss in haplotype diversity. Similarly, the overall data complexity can be reduced even more using the lossy approach when some degree of loss of haplotype diversity can be tolerated. In the example shown, a 10% haplotype diversity threshold loss was used although it will be appreciated that other values may be readily substituted depending upon the desired stringency of analysis. The results shown for Chromosomes 21 and 22 indicate similar findings and demonstrate the overall utility of the data reduction methods.

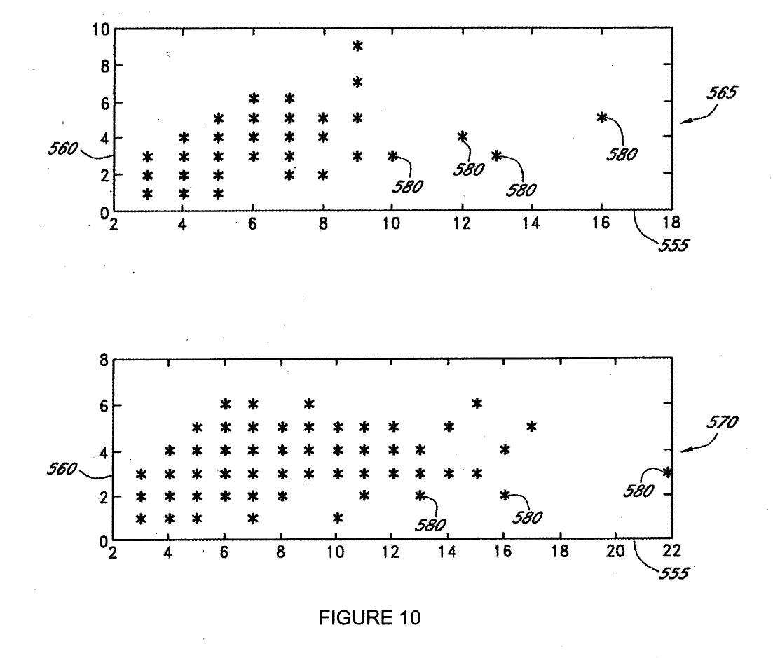

FIG. 10 further illustrates the relationship between the original number of SNPs in an LD block (shown on the horizontal axis 555) and the reduced SNP subset number identified as being useable without loss of haplotyping diversity (shown on the vertical axis 560). The results from both the African-American and Caucasian populations (565 and 570 respectively) demonstrate that in many cases a reduced SNP subset can be identified and used in place of the original larger SNP set. Such improvements can be noted particularity with reference to selected data points 480 wherein the may be a several fold reduction in the number of SNP necessary to represent a LD block.

When evaluated as a whole, the SNP set for the African American population has been reduced by approximately 18% and the Caucasian population reduced by approximately 32% with little or no loss of haplotype distribution information.

It is noted that conventional methods used to find the SNP subset identification typically are generally concerned with complete genes or randomly selected loci, as compared to the present teachings which focuses on LD blocks and block diversity. In conventional methods the number of haplotypes, and more importantly, the amount of information in the haplotype distribution is expected to be much higher and as a result these solutions generally focus on locally optimal solutions. Conversely, the present teachings may be used to compute globally optimal solutions in both lossless and lossy approaches depending on the amount of diversity loss which can be tolerated during the analysis. Thus, the data reduction methods of the present teachings often improve upon and surpass conventional methods for haplotyping analysis and LD block identification.

In one aspect, the two step (phase) data reduction approach of the present teachings provides a means to significantly reduce the amount of data necessary to perform haplotyping analysis. For example, examination of a haplotype block of 22 SNPs using conventional methods necessitates evaluating approximately 4.2 million potential SNP combinations. Using the aforementioned data reduction approaches desirably provides a means to rapidly reduce the original SNP set size to a substantially smaller subset. For example as shown in the example, if a 22 SNP set is reduced to a subset of only 4 SNPs the resulting number of comparisons that need be made will be dramatically reduced as well.

Although the above-disclosed embodiments of the present invention have shown, described, and pointed out the fundamental novel features of the invention as applied to the above-disclosed embodiments, it should be understood that various omissions, substitutions, and changes in the form of the detail of the devices, systems, and/or methods illustrated may be made by those skilled in the art without departing from the scope of the present invention. Consequently, the scope of the invention should not be limited to the foregoing description, but should be defined by the appended claims.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

1. A method for analyzing nucleotide sequence information during haplotyping analysis, the method comprising:
selecting a data superset comprising single nucleotide polymorphism (SNP) information describing a plurality of SNPs, each SNP associated with a plurality of haplotypes, wherein each haplotype is determined by the sequence of SNPs present in the haplotype;
identifying groupings of analogous SNPs from the data superset whose sequences are analogous in two or more haplotypes;
selecting at least one representative SNP from each grouping of analogous SNPs to be included in a reduced data subset; and
performing a haplotyping analysis using the reduced data subset.

2. The method of claim 1 further comprising, selecting at least one non-analogous SNP to be included in the reduced data subset.

3. The method of claim 1 wherein, performing the haplotyping analysis using the reduced data subset substantially preserves haplotype diversity.

4. The method of claim 1 wherein, performing the haplotyping analysis using the reduced data subset requires fewer computations to complete relative to performing haplotyping analysis using the data superset.

5. The method of claim 4 wherein, computational performance during haplotyping analysis is improved using the reduced data subset.

6. The method of claim 1 wherein, the haplotyping analysis comprises discriminating between haplotypes associated with the SNP information.

7. The method of claim 1 further comprising,
identifying at least one diversity subset from the reduced data subset comprising a plurality of SNPs associated with a selected haplotype;
identifying combinations of SNPs selected from the at least one diversity subset and calculating an entropy value for each SNP combination;
identifying a refined diversity subset from the SNP combinations having an entropy value within a selected range and a selected number of SNPs; and
performing the haplotyping analysis using the refined diversity subset.

8. The method of claim 7 wherein, the entropy value for each diversity subset is determined by assessing the relative frequency of occurrence of the selected haplotype.

9. The method of claim 7 wherein, the entropy value for each diversity subset is determined using a Shannon entropy determination.

10. A method for analyzing nucleotide sequence information, the method comprising:
selecting a data superset comprising single nucleotide polymorphism (SNP) information describing a plurality of SNPs, each SNP associated with a plurality of haplotypes, wherein each haplotype is determined by the sequence of SNPs present in the haplotype;
identifying regions of analogous SNP information for each of the plurality of haplotypes;
identifying at least one representative SNP from the analogous SNP information for each region;
forming a reduced data subset wherein at least a portion of the analogous SNP information is excluded from the reduced data subset while haplotype diversity is preserved by inclusion of the at least one representative SNP in the reduced data subset; and
performing the haplotyping analysis using the reduced data subset.

11. The method of claim 10 wherein, identifying analogous SNPs comprises identifying groups of two of more SNPs whose sequences are substantially identical for each of the plurality of haplotypes.

12. The method of claim 10 wherein, identifying analogous SNPs comprises identifying groups of two of more SNPs whose sequences are substantially complimentary for each of the plurality of haplotypes.

13. The method of claim 10 wherein, selecting at least one representative SNP comprises excluding substantially all of the analogous SNP information for each region with the exception of the at least one representative SNP identified from each region.

14. The method of claim 10 wherein, analogous SNPs are identified by comparing the SNP information in a pairwise manner to identify SNPs whose sequence is identical or complimentary in two or more haplotypes.

15. The method of claim 10, wherein performing the haplotyping analysis using the reduced data subset provides similar haplotyping diversity information as the data superset from which it was derived while improving computational performance during haplotyping analysis.

16. The method of claim 10, wherein use of the reduced data subset during haplotyping analysis reduces the computational complexity of performing the haplotyping analysis.

17. The method of claim 16, wherein formation of the reduced data subset reduces the computational complexity of performing of haplotyping analysis by reducing the total number of SNPs to be analyzed.

18. The method of claim 10, wherein the reduced data subset is used in the evaluation of a selected genetic loci.

19. The method of claim 10, wherein haplotyping analysis using the reduced data subset facilitates discrimination between haplotypes with substantially the same degree of specificity as the data superset from which they were derived.

20. The method of claim 10, further comprising:
identifying a plurality of diversity subsets, each comprising one or more SNPs associated with a selected haplotype, by selecting combinations of SNPs associated with the selected haplotype;
calculating an entropy value for each diversity subset and comparing these values to the entropy value determined for the diversity subset containing all associated SNPs;
identifying an refined diversity subset from the reduced data subset having substantially the greatest entropy value and least number of associated SNPs; and
performing the haplotyping analysis using the refined diversity subset.

21. The method of claim 20, wherein the entropy value for each diversity subset is determined as a probability factor defined for each associated haplotype wherein the probability factor describes the relative associated frequency of occurrence of the selected haplotype.

22. The method of claim 21, wherein the entropy value is calculated using a Shannon entropy determination.

23. The method of claim 24, wherein the diversity subset having the greatest number of SNP combinations is used as a threshold for determination of the refined diversity subset.

24. The method of claim 25, wherein the threshold reduces the complexity of calculations in determining the refined diversity subset.

25. A system for analyzing nucleotide sequence information during haplotyping analysis, the system comprising:
a data collection component that provides functionality for selecting a data superset comprising single nucleotide polymorphism (SNP) information describing a plurality of SNPs, each SNP associated with a plurality of haplotypes, wherein each haplotype is determined by the sequence of SNPs present in the haplotype;
a first data analysis component that provides functionality for identifying a plurality of diversity subsets, each comprising one or more SNPs associated with a selected haplotype, by selecting combinations of SNPs associated with the selected haplotype;
a first computational component that provides functionality for calculating an entropy value for each diversity subset and comparing the resulting entropy values to an entropy value determined for the diversity subset containing substantially all associated SNPs;
a second data analysis component that provides functionality for identifying an refined diversity subset from the data superset having substantially the greatest entropy value and least number of associated SNPs; and
a second computational component that provides functionality for performing the haplotyping analysis using the refined diversity subset.

26. The system of claim 25, wherein the first computational component determines the entropy value for each diversity subset using a probability factor defined for each associated haplotype wherein the probability factor describes the relative associated frequency of occurrence of the selected haplotype.

27. The system of claim 25, wherein the first computational component calculates the entropy value using a Shannon entropy determination.

28. The system of claim 27, wherein the second data analysis component identifies the diversity subset having the greatest number of SNP combinations which is used as a threshold for determination of the refined diversity subset.

29. The system of claim 28, wherein use of the threshold reduces the complexity of calculations in determining the refined diversity subset.

30. A method for analyzing nucleotide sequence information during haplotyping analysis, the method comprising:
selecting a data superset comprising single nucleotide polymorphism (SNP) information describing a plurality of SNPs, each SNP associated with a plurality of haplotypes, wherein each haplotype is determined by the sequence of SNPs present in the haplotype;
performing a first data reduction on the data superset by identifying redundant SNPs comprising two of more SNPs whose sequences are identical or complimentary for each of the plurality of haplotypes and removing at least a portion of the redundant SNPs from the data superset;
performing a second data reduction on the data superset by comparing the SNP information in a pairwise manner to identify analogous SNPs whose sequences are identical in two or more haplotypes and removing at least a portion of the analogous SNPs; and
performing a haplotyping analysis using the remaining SNP information in the data superset.

31. The method of claim 30, further comprising performing a third data reduction using the remaining SNP information wherein the third data reduction comprises:
identifying a plurality of diversity subsets, each comprising at least one SNP associated with a selected haplotype, by selecting combinations of SNPs associated with the selected haplotype;
calculating entropy values for each diversity subset;
comparing the calculated entropy values to an entropy value determined for a diversity subset containing substantially all associated SNPs;
identifying a refined diversity subset having substantially the greatest entropy value and least number of associated SNPs; and
performing the haplotyping analysis using the refined diversity subset.

32. The method of claim 31, wherein the entropy value for each diversity subset is determined based upon a probability factor defined for each associated haplotype wherein the probability factor describes the relative associated frequency of occurrence of the selected haplotype.

33. The method of claim 31, wherein the entropy value is calculated using a Shannon entropy determination.
